(19) United States
(12) Patent Application Publication
(10) Pub. No.: US 2013/0011440 A1
(43) Pub. Date: Jan. 10, 2013

(54) METHOD AND DEVICE FOR DEPOSITING THIN LAYERS, ESPECIALLY FOR THE PRODUCTION OF MULTIPLE LAYERS, NANOLAYERS, NANOSTRUCTURES AND NANOCOMPOSITES

(75) Inventors: Franz Herbst, Michelstadt (DE); Valery Serbezov, Plovdiv (BG)

(73) Assignee: VASCOTEC GMBH, Gera (DE)

(21) Appl. No.: 13/340,727

(22) Filed: Dec. 30, 2011

Related U.S. Application Data
(63) Continuation of application No. PCT/DE2010/000739, filed on Jun. 28, 2010.

(30) Foreign Application Priority Data
Jun. 30, 2009 (DE) 10 2009 031 767.8
Jun. 30, 2009 (DE) 10 2009 031 768.6

(51) Int. Cl. C23C 16/48; A61K 9/00; B82Y 5/00

(52) U.S. Cl. 424/400; 427/596; 118/723 R; 977/700; 977/906

(57) ABSTRACT

The present disclosure relates to a method for the deposition of thin layers, particularly for producing multi-layer coatings, nanolayers, nanostructures and nanocomposites by laser deposition from target materials on a substrate surface, which is characterized by the following features: a) the target is divided into segments with materials having most differing physical and/or chemical properties; b) individual segments of said target are irradiated with an in each case different radiation intensity by means of a controlled energetic distribution of the focused laser energy via the laser beam cross section so that each target segment absorbs the quantity of laser energy during the irradiation, which is required to evaporate or desorb the target material present in the respective segment.

### TECHNICAL FIELD

The present disclosure relates to a method and a device for depositing thin layers, especially for the production of multiple layers, nanolayers, nanostructures, and nanocomposites. The disclosure also relates to a substrate comprising a coating based on a composition of organic-inorganic nanocomposites manufactured using the disclosed method and device, in particular for medical and pharmaceutical applications.

### BACKGROUND

The terms “thin layer” and “thin film” in material combinations are used to describe layers with thicknesses ranging from a few nanometers to several microns, which are applied to a carrier material (hereinafter referred to as substrate). Such layers are often crystalline. Thin layers, due to their specific properties, are widely used and not substitutable in various demanding technological products. Thin layers are used in industries such as for example the medical field, bio-technology, in the energy sector, the automotive industry, or in aeronautical and aerospace. The coating of substrates with thin layers, in particular with nanocomposites, often changes a substrate's physical properties. This includes but is not limited to a substrate's thermal, optical, and dielectric properties, its strength, electrical conductivity, etc.

For the deposition of a thin layer, which is usually less than 1 μm thick, materials are applied to a substrate by various methods of thin-film technology to be thereafter processed or structured. The deposition of the layers is mainly performed using physical vapor deposition (PVD) and chemical vapor deposition (CVD). The following discussion is limited to physical vapor deposition.

PVD refers to vacuum-based coating methods of thin-film technology wherein thin layers are formed directly by condensation of a material vapor of the film material. The PVD group includes thermal evaporation, electron beam evaporation, laser beam evaporation (pulsed laser deposition, PLD, pulsed laser ablation), vacuum arc evaporation, Arc-PVD, molecular beam epitaxy, sputtering (cathode evaporation), ion beam deposition (IBD), as well as ion plating.

Common to all these methods is that the material to be deposited is present in solid form in a usually evacuated coating chamber. The material, referred to below as the target, is vaporized by bombarding it with laser beams, magnetically deflected ions or electrons, or by arc discharge.

IBD is usually used for the deposition of ceramic-matrix nanocomposites. IBD provides high-quality deposition of layers at low temperatures (close to room temperature). The disadvantage of this deposition method, however, is that the deposition rate is relatively low and even substrates of simple geometry can require complex manipulation to ensure an even coating.

Sputtering is a physical process in which atoms are ejected from a target due to bombardment with high-energy ions (primarily noble gas ions) and pass into the gaseous phase. Sputtering is a highly flexibility technology. It is capable of coating almost all substrates of very different geometries with a plurality of materials such as metals, alloys and a plurality of other materials. The main advantage of this method lies in the absence of melt and droplet problems. If a magnet is additionally applied under the target, this is called magnetron sputtering. In this configuration, all conductive materials can be deposited. The development of magnetron sputtering yielded larger ion currents or an increase in plasma energy respectively. The disadvantage of this method is that this process only occurs in inert or reactive gases or gas mixtures, and the coatings produced in this way can still contain residues of these gases. It is therefore necessary to ensure a very precise control of the gas flow. A further disadvantage lies in the impossibility of depositing thin layers of organic materials. For this reason magnetron sputtering is usually only used for the deposition of metal-matrix and ceramic-matrix nanocomposites.

Vacuum arc evaporation is one of the ion plating PVD processes. It uses an electric arc between the chamber and the cathode target to melt and vaporize the target material, which is subsequently applied to the substrate. In this process the majority (up to 90%) of the evaporated target material is ionized. Disadvantages of this method are that the arc glow discharge is instable, that the cathode erodes unevenly, and that melt droplets occur, and therefore the quality of the resulting layers suffers as a result. A further disadvantage is that it is not possible to deposit organic materials.

Pulsed laser deposition (PLD) has established and proven itself within thin film technology as a precise method for depositing particularly high quality layers. Here, the material of the target is illuminated with high intensity laser radiation (˜100 MW/cm²). At the point of impingement of the laser beam, a plasma plume is formed, in which plasma propagates at high speeds, the ions reaching energies of around 10-100 eV/ion. The substrate, which is brought to an appropriate temperature, is placed a few centimeters from the target within the plasma plume. Ablated target material is deposited on the substrate and forms a film thereon. The interaction of the laser beam with the target material can be controlled by the controlled application of high energy to low energy interactions. The selected laser interactions with the target material are dependent upon the nature of the target material and are achieved by adjusting the laser parameters or through the appropriate choice of a suitable laser. The disadvantages of this method relate primarily to the relatively slower deposition than with other PVD methods like, for example, electron beam evaporation. There is the possibility of condensation on the substrate and large surface areas cannot be generated. Finally, the deposition of organic materials is made difficult by the possible destruction of the materials.

Further recent developments in the field of the PLD of polymers, biopolymers and organic materials are matrix-assisted pulsed-laser evaporation (MAPLE) and resonant infrared PLD (RIR-PLD). The disadvantage of the MAPLE-based technology is the use of a frozen target (frozen e.g. using nitrogen), containing specific solvents (e.g. dimethoxyethane (DME), toluene), that serve as absorbers for controlled laser energy distribution and thus prevent photochemical damage or fragmentation of the polymer target. The use of frozen targets limits the number of usable polymers that require coating. A further disadvantage is the low coating rate. RIR-PLD uses resonant photochemical reactions which are set to the vibration mode of the target to be evaporated. The disadvantage of this method is the complicated and expensive construction of the reactor for the interaction with the target material like for example the use of a free electron laser and the impossibility of the deposition of nanomaterials.

The continuous compositional spread (CCS) technique is based on the sequential deposition of sub-monomolecular layers of each material from various targets, with the aid of which an atomic-level mixture of the individual materials can be achieved. Adjustment of the respective mixture of the target materials is achieved by adjusting the number of laser pulses that are fired at the target. The disadvantage of this method is the complex apparatus structure of various targets or focusing lens systems and the possibility of depositing only inorganic materials.

U.S. Pat. No. 6,660,343 B2 discloses a method of depositing materials using PLD, MAPLE or MAPLE-DW, in which a target is used by means of which separate segments can be formed in one plane. The disadvantage of this method is that materials with very different physical/chemical properties cannot be used in the individual segments. The reasons for this are the destruction or fragmentation by photochemical processes for materials that require a low energy level for their destruction. Alternatively, two targets with very different physical/chemical properties can be used by applying two different lasers. However, the industrialization of this method and the device is complex and involves considerable expense. U.S. Pat. No. 6,660,343 is hereby incorporated by reference thereto.

Different methods of coating substrates using PLD of segmented targets are described in the publications US 2004/0110042 A1, US 2002/0081397 A1, DE102007009487 A1 and US 2008/0006524 A1, all of which are hereby incorporated by reference thereto. Especially in US 2008/0006524 A1, a PLD method which uses an ultra-fast laser for material ablation of the target material is presented. The disadvantage of this method lies in the use of a multitarget manipulator, which uses diffractive optical elements to achieve an optimal “flat-top” beam profile” and the constant introduction of background gas to optimize the quality of the resulting nanoparticle size and distribution.

EP1101832 B1 describes a process for the combinatory preparation of a library of materials in the form of a two dimensional matrix in the surface area of a flat substrate. The disadvantage of this method is the use of a complex masking technique, which allows a defined deposition of the separate segments on one substrate. In addition, there is a further difficulty within the industrialization of the process.

Furthermore, hybrid techniques like for example PLD magnetron sputtering and laser arc deposition have been developed to produce high quality thin layers, nanostructures and nanocomposites. However, this leaves unresolved the problems of the deposition of organic/inorganic thin layers, nanostructures and nanocomposites of predetermined properties under strict control of all process parameters as well as with the simultaneous or sequential depositions of atomic monolayers up to layers of micron thickness in a vacuum or at atmospheric pressures.

A very promising inorganic material for organic/inorganic nanocomposites is magnesium and its alloys. Magnesium alloys are used for the manufacture of biodegradable stents. Stents are known which are made of a magnesium alloy and are coated with a polymeric pharmaceutical layer. The disadvantage of this design is the use of a polymer which can lead to immunological reactions.

Stents are known, which comprise an inner pharmaceutical layer which is covered by an outer layer made of magnesium. The disadvantage of this method lies in the use of dip coatings and the poor control of layer thickness as well as the impossibility of producing nanocomposites.

For the above reasons, improved methods are needed for producing high quality thin layers, in particular multiple layers, nanolayers, nanostructures and nanocomposites that do not suffer from the limitations described and that allow the possibility of producing a new generation of ultra-thin layers, and have thereby in a position to generate products or materials with a superior surface.

### SUMMARY

A method and a device for laser deposition of target materials in thin layers is disclosed, which allows the production of multiple layers, nanolayers, nanostructures, and nanocomposites made of materials whose physical and/or chemical properties are very different (hybrid nanocomposites). The disclosed method allows the coating of large surfaces, a selective coating of the substrate at predetermined places, coating a substrate with multiple layers. The disclosed method avoids damaging the coating material. It allows building of thin layers having depth-dependent variable material compositions, i.e. the method facilitates substance gradients within the nanocomposites.

In accordance with the invention the objectives above are met by the features of claim 1. Advantageous embodiments of the method and the device of the invention are provided in the dependent claims.

It is well known that the intensity distribution within a focused laser beam is non-uniform. The time-averaged intensity varies both with the radial distance from the center axis of the beam and with the axial distance from the beam's narrowest point (the “waist”). Focused laser beams exhibit a non-uniform beam width. Short-focusing optical systems cause the width of a laser beam to increase more quickly with axial distance from the beam's waist than long focusing systems. The density of the laser energy in this region can vary by several orders of magnitude and this variation is not linear. An ideal laser beam exhibits a rotationally symmetrical Gaussian energy distribution across its beam.

The disclosed method takes advantage of the uniform or non-uniform intensity, i.e. energy distribution, over the cross-section of the laser beam. Conventionally, the target in PLD comprises a single source material. In contrast, the disclosed method provides a target which is segmented into several, at least two target segments. Segments may be regions and/or planes. The segmented target comprises materials of different physical and/or chemical properties in each segment. It may for example comprise a first segment comprising an organic target material, a second segment comprising an inorganic target material, and a third segment comprising a ceramic target material. The interaction of target segments with the non-uniform energy density of the laser is defined and factors influencing the energetic action of the laser beam on the regions of the target are controlled. A successful deposition of different target materials can be realized in one process, using only one target, and one laser beam. The method may even operate with a single laser pulse. In this arrangement, the segmented target may consist of any solid material surface and be of any shape, composition or orientation.

Due to the variation in energy distribution of the focused laser beam along the interaction axis of the laser beam with the target, the interaction of the laser beam with the target is different for each target segment. Each target segment absorbs only as much laser energy as is necessary to vaporize/desorb the target material located in the respective segment without causing destruction, modifications or changing the functionality of the target material.

The present method can produce thin film deposits of both organic and inorganic materials in a single cycle. Target materials that require a relatively lower energy density to vaporize may be placed in target segments which are exposed to the radially outer portions the laser beam and/or axially distant to the waist of the focused laser beam. Target materials that require a relatively higher energy density to vaporize are placed in target segments which are located radially close to the center of the laser beam and/or axially close to the waist of the laser beam. Use of segmented targets permits the synthesis of entirely novel hybrid nanostructures, nanocomposites and entirely new materials with previously unknown properties and their deposition onto substrate surfaces.

Additional factors influencing the energetic action on the target regions can be laser beam energy density, wavelength, pulse duration, number of laser pulses, laser pulse repetition rate, substrate-target distance, target orientation and other known parameters. The process can be carried out in a closed room, specifically a reaction chamber, which provides control over environmental factors surrounding the target and substrate. Control over environmental factors includes e.g. the substrate's temperature or the presence, composition, pressure and temperature of gases within the chamber. The substrate can be cooled or heated. During the process, inert gases, reactive gases or gas mixtures can be fed into the chamber.

In relation to the density of the laser beam energy and the density of the laser power and by fixing all other parameters, various physical processes in the interaction of the laser beam with the target material can be achieved, such as e.g. absorption, heating, heating with evaporation, heating with melting and evaporation, very rapid heating and ablation, or direct ablation. These various processes are used for the successful deposition of individual materials. For example, the required energy density of the laser for deposition in the case of organic compounds or other complex organic materials is very small, and the process must be carried out very carefully, to prevent destroying the functional groups of the organic material and resulting in fragmentation. For ceramics, metals, metal alloys and other inorganic compounds, the required energy density of the laser for the laser plasma and the transfer of target materials on to the substrate as ions, electrons, neutral atoms, clusters, fine grains, drops and similar must be very high. The optimal intensity for the deposition is composed of the photon energy of the laser (or wavelength of the laser), the pulse duration, and the characteristics of the target materials.

The present method is particularly suited to the use of coatings for medical equipment such as implants, chemoselective or bioselective surfaces for sensors, devices in the pharmacy, the energy sector, in aeronautics and aerospace, and also the automotive industry. Examples of such devices are stents, catheters, drug-releasing implants, biosensors, surface acoustic wave devices (ASW), optical waveguides, optical devices, solar cells, tools, ultra hydrophobic and ultra hydrophilic surfaces, among others. Examples of coatings on medical devices are nanocomposites from bio- and haemocompatible polymers as well as pharmaceuticals, nanocomposites of bio- and haemocompatible polymers and ceramics, nanocomposites of biodegradable polymers and pharmaceuticals, nanocomposites of biodegradable metals and pharmaceuticals, and so on. Examples of chemoselective materials are described in detail in “Choosing polymer coatings for chemical sensors” (CHEMITECH, Vol. 24 No 9, pp 27-37, 1994 McGill et al.). Also of interest are nanocomposites of ceramics, dendrimers, and DLC (diamond-like-carbon). Examples of bioselective materials include proteins, peptides, antibodies, DNA, RNA, polysaccharides, lipids and others as well as their metal-, ceramic- or polymer nanocomposites.

The disclosed method can be used to produce a substrate comprising a coating based on a composition of organic-inorganic hybrid nanocomposites. Such a substrate may e.g. be used for medical and pharmaceutical purposes. The physical and/or chemical properties of the hybrid nanocomposites may be very different. Such organic/inorganic materials with predetermined properties may be produced in one technological cycle. Parameters like uniformity, homogenous thickness, coating of predetermined areas and “surface coverage” must be exactly controllable.

The disclosed substrate is characterized in that the coating composition exhibits a layer which consists of biodegradable inorganic and/or organic nanocomposites and releases an active ingredient. In particular, the active ingredient may be a pharmaceutical. Preferred pharmaceuticals may be those known to the person skilled in the art and described in §2, section 1 of the German Drug Law. Preferred pharmaceuticals may also be rapamyzin and paclitaxel. Generally, the disclosed substance may comprise any long-term or immediately-acting pharmaceutical.

The drug-releasing layer is constructed from a biodegradable material that does not trigger immunological reactions in the body even during its degradation. Suitable materials are certain metals, metal oxides and their alloys or other inorganic or organic compounds. The inorganic material of the substrate coating for organic/inorganic hybrid nanocomposites is preferably a metal, in particular magnesium or its alloys.

The drug-releasing layer preferably has mechanical properties that ensure a sufficiently high abrasion resistance of the layer during the passage of the implant to its destination, and a sufficiently high resistance to stress—as are required in the case of a stent during its expansion in a stenosis. Here, there must be an intact, undamaged surface of the implant that will permit a homogeneous release of the pharmaceutical. Surfaces constructed from polymers, such as polylactides, do not possess the necessary resistance to abrasion or to stress.

The disclose method can be used in a device by means of which, through a controlled energy distribution of the focused laser energy over the beam cross-section, individual segments of the target can be irradiated each with a different radiation intensity.

### BRIEF DESCRIPTION OF THE DRAWINGS

FIG. 1 is a schematic representation of a method and of the device for depositing thin layers.

FIG. 2 shows a lateral and perspective view of an exemplary segmented target.

FIG. 3 shows a lateral and perspective representation of an alternative target comprising 4 different segments.

FIG. 4 is a schematic representation of parts of a target divided into four segments.

FIG. 5 is a schematic representation (cross section) of a target showing the distribution of the laser energy in the plane of the focused laser beam.

FIG. 6 is a schematic representation of the Gaussian energy distribution of the laser energy in the plane of the focused laser beam for various materials.

FIG. 7 to 9 show alternative arrangement of a segmented target which is disposed behind a segmented polarization plate with a polarizing filter.

FIG. 10 an examplary representation of the results of energy-dispersive X-ray spectroscopy (EDS).

FIG. 11 is an alternative example as shown in FIG. 10.

FIG. 12 is an exemplary representation of the results from Fourier transformation IR spectroscopy.

FIG. 13 is an alternative example of FIG. 12.

### DETAILED DESCRIPTION

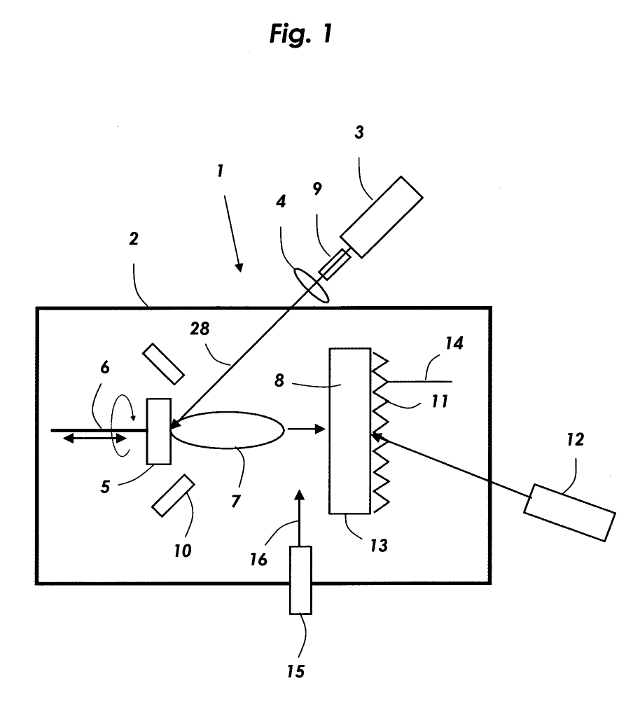

FIG. 1 shows a schematic representation of a method and a device 1 for depositing thin layers. The device 1 comprises a deposition chamber 2 and at least one laser 3, preferably a pulsed laser 3, which is focused on a segmented target 5 via an adaptive optical system 4. Adaptive optical system 4 may comprise various optical elements such as lenses, mirrors, prisms, filters, and tuners. The target 5 is mounted at or on a movable carrier 6, which allows a translational and/or a rotating movement of the target 5. The target 5 typically rotates at about 0.05-3000 Hz.

A substrate holder 13 is provided and preferably electrically insulated. A substrate 8 is placed onto substrate holder 13. The temperature of the substrate 8 can be controlled to maintain a predefined temperature with a conventional substrate heater and/or cooler 11, which is disposed at the back of the substrate 8. The substrate 8 can alternatively or additionally be heated with a heating laser 12. The temperature of the substrate 8 is measured by a thermocouple 14 or by other suitable means. The use of a heating laser 12 instead of or in addition to the substrate heater and/or cooler 11 supports the formation of nanocomposites having different local structures within the layer. Heating laser 12 allows selective heating of only parts of the substrate 8. Parts of the substrate 8 which are exposed to local heating exhibit the formation of crystalline or polycrystalline structures. Parts of the substrate 8 which are not exposed to local heating exhibit the formation of amorphous structures. The preferred substrate temperatures depend upon the desired type of the substrate 8 and the type of coating material. For nanocomposites, consisting, for example, of a ceramic polymer, specifically of DLC-Teflon, the target temperature is preferably between 25-60° C. For nanocomposites consisting of metallic rhodamine, the target temperature is preferably between 25-50° C. For nanocomposites consisting of metal and organic compounds the target temperature range is preferably between 25-250° C.

Deposition chamber 2 may be evacuated and used as a vacuum chamber. Alternatively, a gas inlet 15 permits the entry of gases 16 into deposition chamber 2. The deposition chamber 2 may operate at reduced pressure with the addition of an inert gas, a reactive gas or a gas mixture.

The angle of incidence between the laser 3 which generates laser beam 28 and the target 5 may be adjustable and is typically 45°. The laser beam 28 may be guided relative to the target 5 by use of a scanner 9.

Laser 3 may be selected from a variety of suitable technologies. Commonly used is a pulsed laser, especially a short-pulsed laser such as a UV laser or a laser operating with the visible wavelength. Laser 3 may for example be an excimer laser for the generation of electromagnetic radiation in the ultraviolet wavelength range. It may also be a nitrogen laser or other short-pulsed laser selected from the group consisting of Nd:YAG-lasers (neodymium-doped yttrium aluminium garnet), Nd:YLF-lasers (neodymium-doped yttrium-lithium-fluoride-laser), CVL (Cooper vapor laser), ps laser (picosecond laser), fs laser (femtosecond laser), fiber lasers, and CO2 lasers (carbon dioxide lasers). Suitable lasers usually emit light at a wavelength of 193 nm-1200 nm with an energy density of 20 mJ/cm² up to 15 J/cm² (typically 50 mJ/cm²-5 J/cm²) and a pulse duration of from 10⁻¹² to 10⁻⁶ seconds and a pulse rate between 0 and 30 Hz. In general, the energy density influences the different regimes of interaction, morphology and topology of the layer surface.

The distance between the target 5 and the substrate 8 is typically between 2-20 cm and preferably about 8 cm. In general, larger distances are more suitable for the coating of larger surfaces. The target-substrate distance is inversely proportional to the layer thickness achieved during a given period of deposition.

The target 5 and the substrate 8 are positioned in a closed environment such as deposition chamber 2. The environmental factors of the substrate 8, such as temperature, pressure and material on the segmented target 5 are controlled in order to achieve an optimal coating process. By this, fragmentation or derivativisation of the coating material is eliminated or minimized. Suitable environments for the coating can be argon, oxygen, helium, nitrogen, alcohols, hydrocarbons or corresponding gas mixtures. Other non-reactive gases can be used as a substitute for argon. The pressure inside the deposition chamber 2 during the coating process can reach between 10⁻⁴ and 760 torr.

In a preferred embodiment of the device 1, material injectors 10 are provided in the deposition chamber 2 near the target 5. These material injectors 10 operate by injecting materials during the coating process, either continuously or synchronously pulsed with the repetition rate of pulses from laser 3. Materials may be injected in various states, for example gases, gas mixtures, pills, liquids or combinations thereof. Material may be injected directionally parallel to the target 5, above the target 5, or in the direction of the substrate 8. The choice of arrangement determines the degree of the fluid situation of the evaporating material from the target 5. The distance between target 5 and substrate 8 is selected on the basis of the selected injected material, and must ensure that only the evaporated target material strikes the surface of the substrate 8. All possible reactions to the cooling of the plasma, the recombination process and the physical elimination of the injected material can occur in the area of the substrate 8. The physical removal of the injected material to fluidize the required substance is accomplished with a vacuum pump. In a particular example helium/argon gas has been found to be a suitable injection material for the production of ceramic-metal nanocomposites like DLC-Ag or DLC-Pt or DLC-Ag+Pt nanoparticles.

The thickness of the coating film is generally proportional to the number of laser pulses, or the time of the coating process. The film thickness can be adjusted by the number of laser pulses, the target temperature, the distance between the target 5 and the substrate 8 and the laser energy density. The usual thickness for the production of ceramic-metal nanocomposites is between 70 nm and 200 nm.

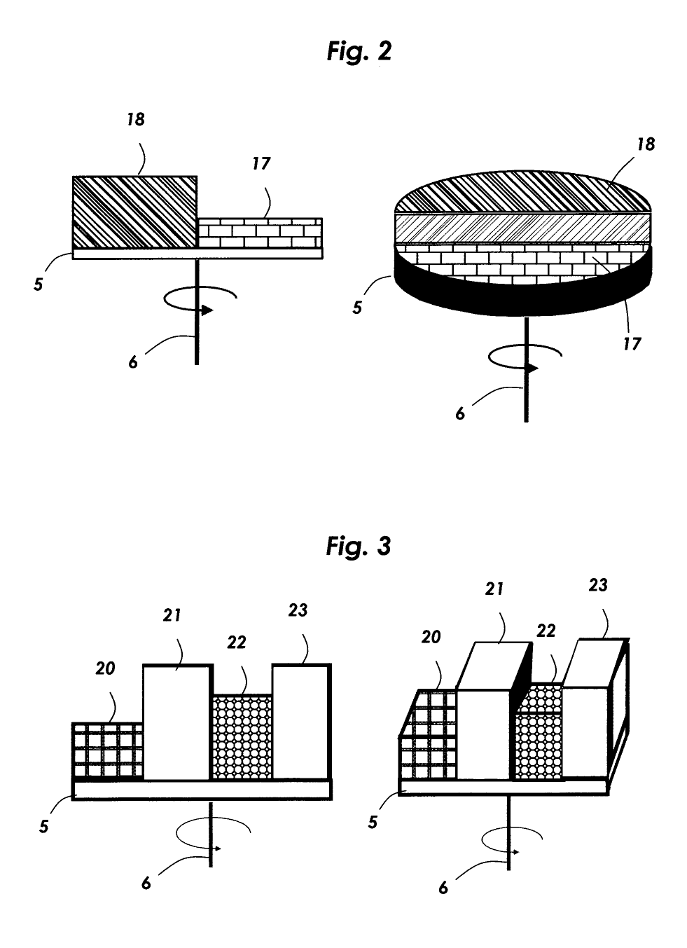

Referring now to FIG. 2, an exemplary target 5 which is segmented in two planes (17, 18) is shown schematically. A first segment 18 of the targets 5 comprises an organic material and a second segment 17 comprises an inorganic material. While target 5 is shown with just two segments 17 and 18 there is generally no upper limit as to how many segments target 5 comprises. The number of segments can vary according to the application. Target 5 can have any shape. It may e.g. be parallelepiped, pyramidal, cuboid, spherical or assume other complex shapes. The material on the segments 17, 18 may be an alloy or a composite.

Use of a segmented target 5 in combination with a properly aligned laser 3 allows the creation of thin nanocomposite layers on substrate 8 with non-uniform material composition. The relative ratio of an organic and an inorganic component within the thin nanocomposites layer on substrate 8 may for example gradually change with increasing depth from the layer's surface. At its surface the layer may contain a higher relative content of the inorganic material than deeper within the layer. Non-uniform material composition within a thin layer is achieved by controlling the deposition rate of two of more target materials over time. For example, the deposition rate of an inorganic component deposited on the substrate surface may be high initially, and decrease towards the end of a deposition cycle. In contrast, the deposition rate of an organic component deposited on the substrate surface may be low initially, and increase towards the end of a deposition cycle. By changing the deposition rate of two or more target materials over time, various material gradients by depth within the resulting thin layer on the substrate 8 can be achieved.

By rotating target 5 the first target segment 17 and second target segment 18 are alternately exposed to the laser beam 28. This generates a plasma plume with alternating composition from the two target materials. This can be used to alternately deposit complex organic compounds and inorganic materials on the substrate 8. In the case of complex organic compounds, a low energy process is carried out non-destructively for the labile substances to be transferred. In the second process a laser ablation is performed.

A rapid rotation of the target 5 produces a single nanocomposite layer comprising the materials of the individual segments 17 and 18. If the rotation is slow, a multi-layer-nanocomposite consisting of alternating layers of the different materials from the individual target segments 17 and 18 is created.

Segments 17 and 18 of the target 5 can also be arranged in such a way, rotated, or translationally moved that their position varies synchronously or asynchronously with pulses of laser 3.

Alternatively or in addition to moving the target 5 the substrate 8 may also rotate, translate or be moved in other ways during the coating in order to ensure the uniform coating of otherwise difficult to coat complex three-dimensional object surfaces.

Referring now to FIG. 3, a segmented target 5 can also be used for the production of thin multilayers. Segmented target 5 as shown is used in a dynamic operational mode. The rotating target 5 comprises four segments: A first segment 20 comprises an organic material; a second segment 21 comprises a metal; a third segment 22 comprises a ceramic; and a fourth segment 23 comprises a metal.

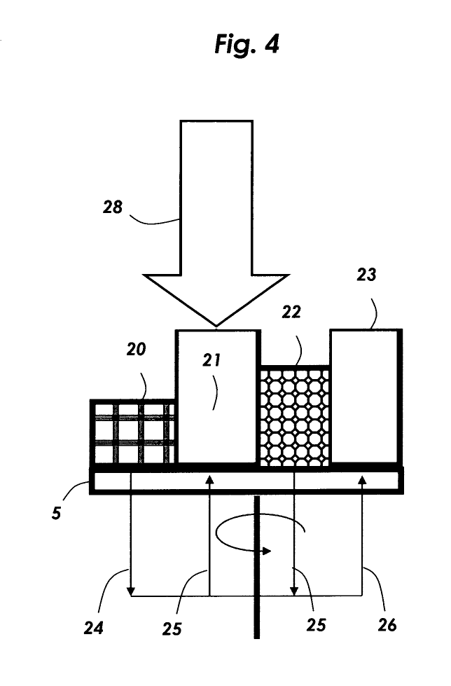

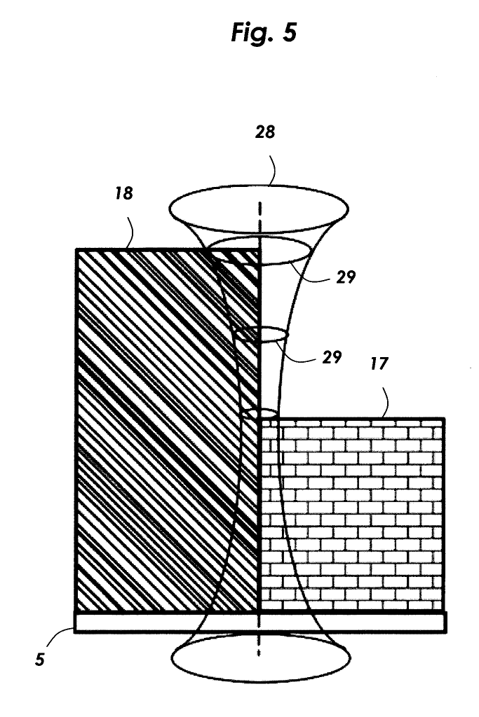

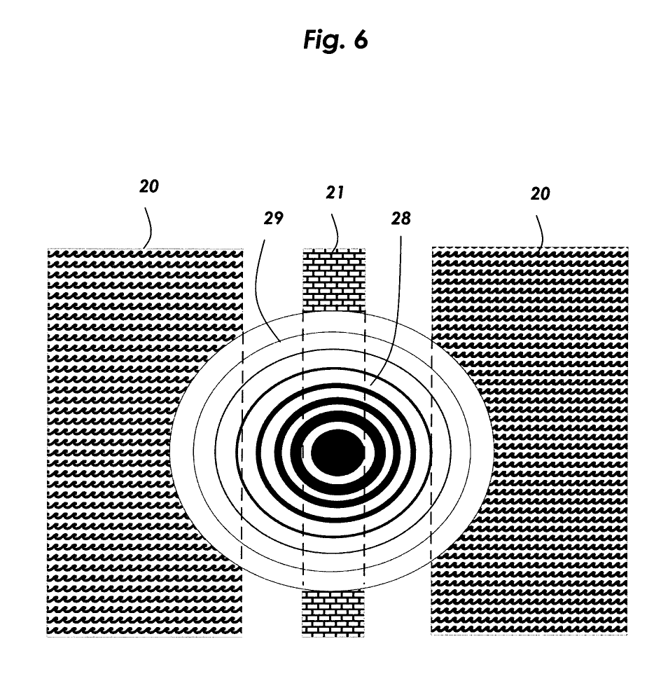

Depicted in FIG. 4 is a segmented target 5 comprising four segments 20, 21, 22, and 23. Each segment is translationally movably attached to an attachment arm. The first segment 20 is operatively connected to a first attachment arm 24. The second segment 21 and third segment 22 are operatively connected to a second attachment arm 25. The fourth segment 23 is operatively connected to a third attachment arm 25. Each attachment arm 24, 25, and 26 can move translationally. As indicated by arrows in FIG. 4, the attachment members are preferably configured to move back and forth in the direction of laser beam 28. Translational movement of target segments 20, 21, 22, and 23 is preferably synchronized with the pulse repetition rate of laser beam 28.

The target 5 can rotate at a uniform rate, variably, or stepwise. During the rotation of the target 5, each segment 20, 21, 22, 23 is alternately exposed to the focused laser beam 28, synchronized with the laser pulses and with the laser beam plane, in which the laser energy density is optimal for the interaction of the respective target material on the selected segments 20, 21, 22, 23. This results in alternating plasma plumes of organic material 20, metals 21, 24 and ceramic 23 to be generated by laser beam 28, which leads to alternating material deposits on the surface of the substrate 8. If the target 5 is displaced stepwise in rotation, the regime of simple multitargets is realized. If the target 5 rotates slowly, then a multilayer composite of different layers of organic material, metal and ceramic is created. If the target 5 rotates quickly, a multicomposite of organic material, metal and ceramic is created. The target 5 can rotate in one technological cycle in the three above-mentioned operational modes and consist of alternate layers of individual composites, multilayers and nanocomposites. Each individual segment 20, 21, 22, and 23 can be of any desired shape, for example, parallelpiped, pyramidal, cuboid, spherical or any other complex shape. The movement of the target 5 and the attachment arms 24, 25, and 26 can be controlled via a pre-set program. This allows the synthesis of nanocomposites with exactly defined properties.

A schematic illustration (cross-section) of the distribution 29 of laser energy in the plane of the focused laser beam for different materials 17, 18 is depicted in FIG. 5. It shows an organic material 18, an inorganic material 17, the focused laser beam 28, and the plane of the laser energy distribution 29.

The inherent characteristic of that laser light being polarized can be used to control the fluence (energy density) of the laser light. An optical filter, preferably a polarizing filter 32, can be placed between the laser source and the target to precisely control the fluence of a laser beam 28, before it reaches target 5. The optical filter can be used to vary fluence of laser beam 28 between 0% and 100%, depending on the position of the filter relative to the axis of the laser beam. Thus it is possible to carry out precise control of the fluence on a target.

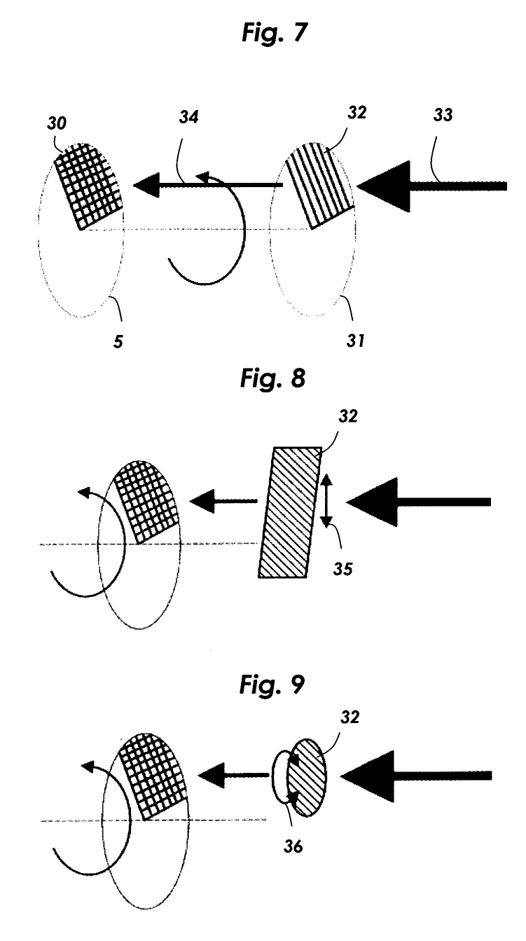

FIGS. 7 through 9 show a segmented target 5, which is located behind a segmented polarization plate 31 with a polarizing filter 32 in different order and configuration forms.

As depicted in FIG. 7, the target 5 is segmented into circular segments 30. Various materials requiring different vaporization energies may be arranged in such circular segments 30. To adjust the vaporization energies exactly for the different target materials, a second disc 31 comprises polarizing filters 32 is positioned on the same axis of rotation as target disc 5. The second disk 31 mirrors the segmentation of the target 5. At points in target 5, which require a lower vaporization energy, the corresponding polarization plate 31 has a polarizing filter 32, which is set to pass only the required vaporization energy. Target disc 5 and polarizing filter disc 31 are synchronized with each other in their rotational movement, i.e. the polarization filter arrangement is identical with the segment arrangement on the target 5. The rotation of the target 5 is synchronized with the rotation of polarizing filter disc 31. This causes the fluence of the laser beam 34 to be a varying fraction between 0 and 100% of the fluence of laser beam 33. Fluence of laser beam 34 is configured to match the need for vaporization energy of the materials in the segmented target 5. The polarizing filters 32 are previously set so that the required fluence attenuation is reached.

Referring to FIG. 8, a polarizing filter 32 is arranged in the axis of the laser beam 33 which attenuates the fluence at exactly the moment when the attenuation is required. Here, a plurality of polarizing filters can be used, whose fluence attenuation has been previously matched to the target material. They are placed in the path of the laser beam 33 at a point in time when fluence attenuation is desired to create attenuated laser beam 34. Polarizing filter 32 in this example describes a translational movement.

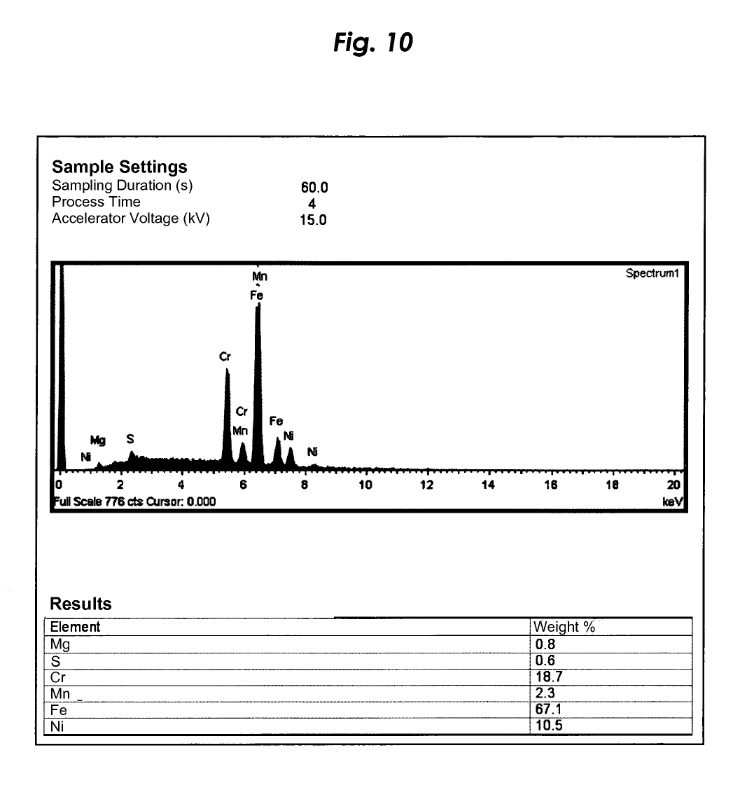

In another example as shown in FIG. 9, a rotating polarizing filter 32 can be placed in the axis of laser beam 33 to create attenuated laser beam 34. As illustrated, polarizing filter can be rotated clockwise and counterclockwise about its rotational axis to attenuate the fluence of the laser beam 33. The desired level of attenuation is achieved by controlling the alignment of polarizing filter 32 with the polarization plan of laser beam 33 at the time of a laser puls.

The use of the disclosed method and device to obtain thin layers with a particular characteristic is further illustrated with reference to the following examples:

### Example 1

To synthesize an organic-metallic biodegradable nanocomposite, a target 5 with a radius of 1.5 cm was produced with segments consisting of a magnesium alloy, and rhodamine 6G. A circular segment comprising one third of the round target 3 consisted of rhodamine 6G (an organic fluorescent dye) having a layer depth of 2 mm. A circular segment comprising the remaining two thirds of the target 5 consisted of magnesium with a layer depth of 3 mm. Both circular segments were fixed onto the target holder 6 which was placed into a deposition chamber 2. The deposition chamber 2 was evacuated to a pressure of 2×10⁻⁴ torr. A TEA Nitrogen (N2) laser with a wavelength of 337.1 nm, a pulse duration of 6 ns and an energy per pulse of 10 mJ and a repetition rate of up to 120 Hz was aimed at the target. The substrate 8 consisted of a rectangular 2×2 cm stainless steel disc 316L. The substrate temperature during the process was 22° Celsius. The distance between the substrate 8 and the target 5 was 5 cm. The total pressure during the process was 5 m ton, the repetition rate of the laser pulse 15 Hz. The energy density at the rhodamine 6G segment was 0.25 J/cm² and on the magnesium segment 3 J/cm². The rotation speed of the target was 200 Hz. The duration of the coating process was 20 min. The thickness of the resulting nanocomposite of magnesium alloy and rhodamine 6G was 250 nm. The nanocomposite produced in this way was examined by scanning electron microscopy (SEM), EDS, fluorescent microscopy and Fourier transform IR Spectroscopy (FT-IR). The EDS results are illustrated in FIG. 10.

### Example 2

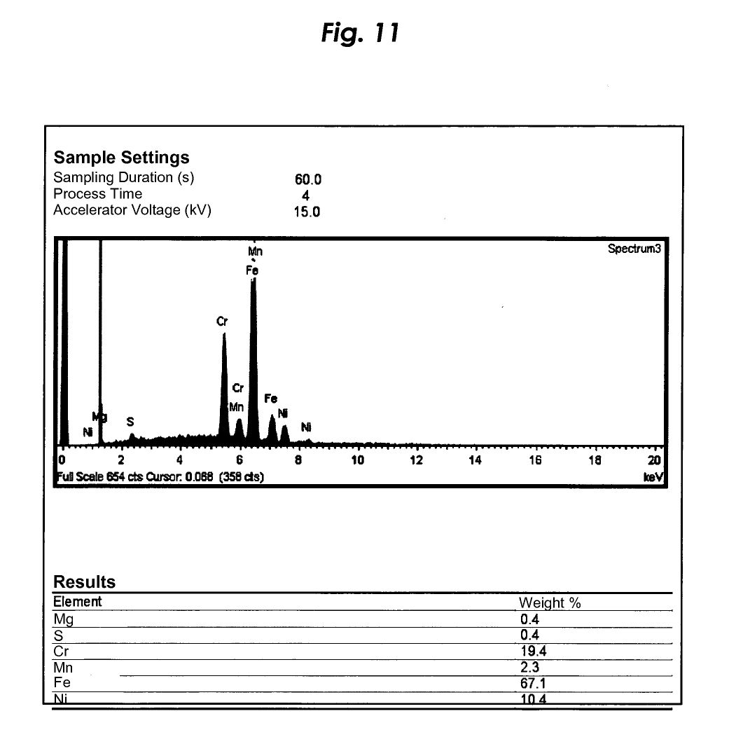

The experiment was conducted in essentially the same way as example 1 above, but here the target consisted of two thirds rhodamine 6G and one third magnesium. It was examined in the same way with SEM, EDS, fluorescent microscopy and FT-IR. The EDS results are shown in FIG. 11.

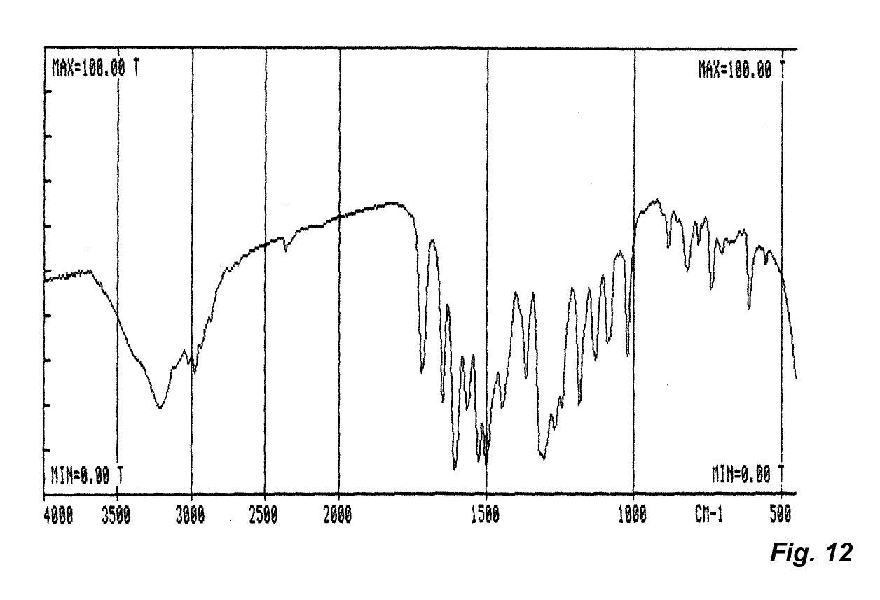

FIG. 3: The experiment was conducted essentially in the same way as example 1 above. Here, the substrate was a round KCl disc with a diameter of 4 cm. The target consisted of only rhodamine 6G, and the energy density of the rhodamine at the target was 0.25 J/cm². It was examined in the same way with SEM, EDS, fluorescent microscopy and FT-IR. FT-IR results are illustrated in FIG. 12.

### Example 4

The rhodamine 6 G was dissolved in methanol and applied to a KCl monocrystal for FT-IR examination and the methanol was evaporated at RT. The target thus produced was used as a reference target for the FT-IR examinations. FT-IR results are illustrated in FIG. 12.

Results: The selection of rhodamine as the organic component in the organic/metallic nanocomposite was made because the distribution of the organic fluorescent dye in the nanocomposite can easily be detected by fluorescence microscopy. If the distribution of the organic dye in the nanocomposite were non-homogenous, then cluster fluorescence would be observed.

In the SEM examinations of the thus prepared coated substrates from experiments 1 and 2, a homogeneous nanocomposite structure could be seen. The individual composite structures had dimensions of around 200-300 nm. Analysis of the element magnesium yielded, in the EDS in experiment 1, a concentration of 0.8% by weight (FIG. 12) and in experiment 2, a concentration of 0.4% by weight (FIG. 11). Since these concentrations were measured on the stainless steel substrates, an accurate conclusion about the concentration of magnesium could not be drawn. However, a decrease was seen in the magnesium concentration in samples from example 2, which suggests a reduced concentration of the proportion of magnesium in the nanocomposite.

In the examinations of the coated substrates from experiment 1, using fluorescent microscopy, a highly homogeneous fluorescence could be seen over the entire surface of the rhodamine/magnesium nanocomposite. No fluorescent clusters were found, which represents evidence of the complete homogeneous distribution of the rhodamine 6G dye in the rhodamine 6G/magnesium nanocomposites. The same distribution was found for the substrates in example 3, which were coated only by laser with rhodamine 6G.

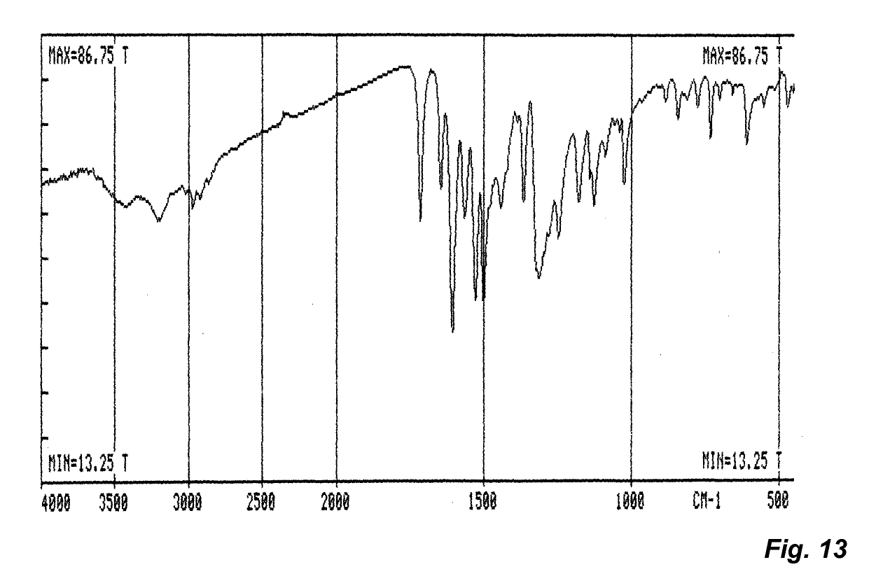

No significant differences in the spectrogram could be detected in the comparison of the FT-IR spectra of dip-coated samples from example 4 (FIG. 13) and laser-coated samples from example 3 (FIG. 12).

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations and broad equivalent arrangements that are included within the spirit and scope of the following claims.

What is claimed is:

1. A method for depositing thin layers by laser deposition of target materials onto a surface of a substrate, comprising the steps of:
a) providing the substrate;
b) providing a target, the target comprising at least two target segments, each target segment comprising a target material having distinct physical or chemical properties;
c) providing a focused laser beam, the focused laser beam having a non-uniform intensity across its beam and a non-uniform beam width along its axial extension;
d) aiming the focused laser beam at the target such that the at least two target segments are exposed to different laser intensity; and
e) coordinating the target materials and the laser intensity such that each target material absorbs an amount of laser energy that is suitable to evaporate or desorb the respective target material.

2. A method as in claim 1, further comprising the step of rotating the target.

3. A method as in claim 1, further comprising the step of moving the target or at least one of the target segments translationally.

4. A method as in claim 1, further comprising the step of rotating the target and translationally moving the target or at least one of the target segments.

5. A method as in claim 1, further comprising the step of rotating or translationally moving the substrate.

6. A method as in claim 1, wherein the at least two target segments comprise a first target segment comprising an organic target material and a second target segment comprising an inorganic target material.

7. A method as in claim 1, wherein the at least two target segments comprise a first target segment comprising an organic target material, a second target segment comprising an inorganic target material, and a third target segment comprising a ceramic target material.

8. A method as in claim 1, wherein the target material comprises an alloy or a composite.

9. A method as in claim 1, further comprising one or more of the following steps:
configuring the target segments to assume a predetermined position to the laser beam;
configuring the non-uniform beam width along its axial extension of the laser beam;
selecting a wavelength of the laser beam;
controlling a pulse duration of the laser beam;
controlling a number and repetition rate of pulses of the laser beam;
selecting a distance of the substrate from the target;
selecting an orientation of the substrate relative to the target.

10. A method as in claim 1, further comprising the step of providing a gas or gas mixture which surrounds the target and the substrate.

11. A method as in claim 1, further comprising the step of providing an injection material which is injected into the target, either continuously or, synchronously with a repetition rate of the laser beam.

12. A method as in claim 11, wherein the injection material is helium or argon or a mixture thereof.

13. A method as in claim 1, further comprising the steps of pulsing the laser beam and moving the target synchronously with the pulsing of the laser beam.

14. A method as in claim 1 wherein the laser beam has a fluence, further comprising the steps of:
providing an optical filter configured to attenuate the fluence of the laser beam between 0% and 100%; and
adjusting the fluence of the laser beam by positioning the optical filter relative to the laser beam.

15. A method as in claim 14, wherein the optical filter is a polarizing filter.

16. A method as in claim 14, further comprising the step of synchronously rotating or translationally moving the target while positioning the optical filter.

17. A method as in claim 14, wherein the optical filter comprises a plurality of filter segments, the plurality of filter segments corresponding to the at least two target segments.

18. A device for depositing thin layers by laser deposition of target materials onto a surface of a substrate, comprising:
a laser;
a reaction chamber;
a target carrier located within the reaction chamber and in sight of the laser; and
a substrate holder within the reaction chamber, wherein the substrate holder is configured to allow translational movement of the target within the reaction chamber.

19. A substrate manufactured by the method as in claim 1, the substrate coated by a thin film of organic-inorganic hybrid nanocomposite, wherein the thin film comprises a non-polymeric layer composed of biodegradable nanocomposites which releases active ingredients.

20. A substrate as in claim 19, wherein the organic-inorganic hybrid nanocomposite comprises a metal.

21. A substrate as in claim 19, wherein the active ingredient is a pharmaceutical.

22. A substrate as in claim 19, wherein the non-polymeric layer composed of biodegradable nanocomposites which releases active ingredients is resistant or abrasion and stress.
